(19) United States
(12) Patent Application Publication
(10) Pub. No.: US 2003/0054030 A1
(43) Pub. Date: Mar. 20, 2003

(54) Method and compositions for the treatment of pruritus

(76) Inventor: Gary Gordon, Highland Park, IL (US)

Correspondence Address: KATTEN MUCHIN ZAVIS, Attention: Patent Administrator, Suite 1600, 525 West Monroe Street, Chicago, IL, 60661-3693, US

(21) Appl. No.: 09/951,490

(22) Filed: Sep. 13, 2001

(51) Int. Cl. A61K 31/485; A61K 9/20

(52) U.S. Cl. 424/465; 514/282

(57) ABSTRACT

A composition for treating pruritus, comprising a compound selected from the group consisting of opioid receptor antagonists, opioid receptor agonists/antagonists, and pharmaceutically acceptable salts thereof, and a compound useful in treating the cause of the pruritus. This invention also relates to a method of treating pruritus using such compositions, and a method for preparing these compositions.

### BACKGROUND OF THE INVENTION

1. Field of the Invention

The present invention relates to a method and composition for treating pruritus by administering one or more of an opioid receptor antagonist, an opioid receptor agonist/antagonist, and pharmaceutically acceptable salts thereof, in combination with one or more compounds useful in relieving itching or treating the underlying cause of the pruritus. The composition may be administered in a variety of forms. When administered topically, the composition may include a multilamellar lipid vesicle delivery system. The method and composition are highly effective in relieving pruritus caused by a variety of underlying pathologies.

2. Related Art

Pruritus, or itching, is an unpleasant symptom that may be related to a plethora of causes ranging from dry skin, systemic disorders, and organ failure; or skin involvement with infectious, allergic, immune, neoplastic, or inflammatory processes; or use of various drugs. Because of the many possible causes of pruritus, the symptoms should be explored to determine the underlying problem. Pruritus is commonly exhibited by individuals suffering from chronic renal disease, cholestatic liver disease, primary biliary cirrhosis, and posthepatic obstruction, as well as diabetes, thyrotoxicosis, AIDS, Hodgkin's disease, lymphoma, and leukemia, for example. Secondary pruritus caused by drugs is common in individuals receiving opium derivatives, such as morphine (see structure, below)

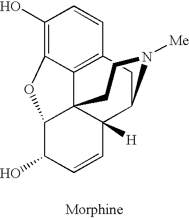

phenothiazines, tolbutamide, erythromycin estolate, anabolic hormones, estrogens, progestins, testosterone, aspirin, quinidine, biologics such as monoclonal antibodies, and vitamin B complexes, for example. Pruritus is also caused by allergic reactions to topical allergens such as detergents, plant products, insect bites/toxins, as well systemic exposure to foods and other allergens. Topical infections with fungal agents are also frequently present, and are complicated by pruritus.

The etiology of the desire to itch is incompletely understood, and effective treatments are elusive. The sensation of itch and the subsequent behavior of scratching, which is a physiologic response to the sensation of itch, can cause significant discomfort for individuals and can, if severe, compromise the skin's effectiveness as a protective barrier. Mechanisms of pruritus have been hypothesized from studies of pain, with which it may share common molecular and neurophysiological mechanisms. It is believed that itching sensations result from the activation of free nerve endings at the dermal-epidermal junction. Stimulation can result from the localized release of histamine (resulting in urticarial wheals and intense itching), as well as other mediators such as vasoactive peptides, enkephalins, substance P, and prostaglandins. Factors including psychological stress, tolerance, presence and intensity of other sensations and/or distractions may determine the degree of itch sensitivity. Dryness of the epidermis and dermis are believed to enhance the sensation of the itch, as well as anoxia of the tissue, dilation of the capillaries, irritating stimuli, and psychological responses. The itch impulse is transmitted from peripheral nerves to the dorsal horn of the spinal cord, across the cord via the anterior commissure, and ascends along the spinothalamic tract to the laminar nuclei of the contralateral thalamus. Thalamocortical tracts of the tertiary neurons are believed to relay the impulse through the integrating reticular activating system of the thalamus to several areas of the cerebral cortex. The scratch response is a spinal reflex can provide temporary relief from itching, but as with other physical stimuli, may result in an undesired cycle of itch, scratch, itch.

The role of the opioid system in the itch/scratch complex is well established. Scientific and other literature describes the induction of scratching behavior due to the administration of opioids. Also, the reversal of itching by the systemic or topical use of compounds that are opioid receptor antagonists or opioid receptor mixed agonist/antagonists has been described in a number of clinical settings, including hepatic disease, insect bites, and itch due to cutaneous T-cell lymphoma. A compound that is an antagonist counteracts the effects of another drug or group of drugs. A compound that is a mixed agonist/antagonist has both the same or similar effects of another drug or group of drugs, and to some extent also counteracts those effects. Examples of compounds that counteract the effects of opioids are shown below:

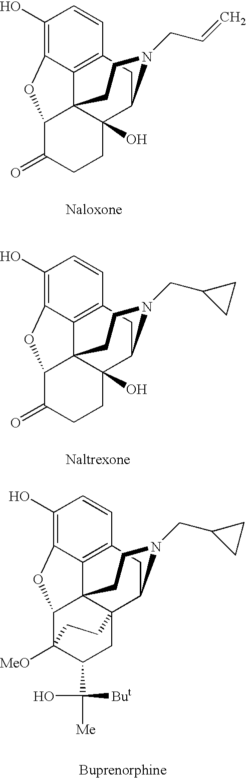

Both peripheral and central mechanisms appear to play a role in the relief of pruritus and these mechanisms have been exploited in the control of clinical symptoms. Systemic and topical anti-histamines, topical and systemic corticosteroids, topical agents such as menthol, and topical and systemic use of naloxone, and kappa antagonists have been used to relieve pruritus. Use of topical emollients, moisturizers, and creams may also be employed. Antibiotics may reduce pruritic symptoms associated with infections. Sedative and tranquilizing agents may also be effective in relieving pruritus of unknown origins, especially if relief is not provided by other agents. Sequestrants may be used to relieve pruritus associated with renal or hepatic disease by binding and removing pruritogenic substances.

Naloxone and related compounds are commonly used to control pruritus induced by administration of opiates. Naloxone is also useful in relieving pruritus having origins not related to use of narcotics, but the effectiveness of these treatments can vary. Other compounds may be useful in relieving pruritus if the mechanism of action of the compound addresses the underlying cause of the pruritic symptoms. However, none of the methods or compositions discussed above address the problem of treating pruritus by administering generally effective opioid receptor antagonists, and/or opioid receptor mixed agonist/antagonists, and/or pharmaceutically acceptable salts of these, in connection with compounds that are directed toward alleviating the underlying cause of the pruritic symptoms. Further, none of those methods or compositions provide for the topical application of the composition using a multilamellar lipid vesicle delivery system.

### SUMMARY OF THE INVENTION

While relief from pruritus is partially achievable using single agents, combinations that impact multiple etiologies provide more effective and sustained relief. These additional agents may impact other pathways related to itching such as local histamine release and inflammation; or the underlying etiology of the clinical condition itself. The nature of this invention is the use of combinations of naloxone, naltrexone or other related opioid receptor antagonists, opioid receptor mixed agonist/antagonists, and pharmaceutically acceptable salts thereof, in combination with other agents for the control of pruritus. Such agents include antihistamines (e.g., diphenhydramine, chlorpheniramine); anti-inflammatory corticosteroids (e.g., hydrocortisone, prednisone, prednisiolone); topical anti-infectives and anti-fungals (e.g., ketoconazole, miconazole, tolfinate), antibacterial agents (e.g., penicillin, ampicillin, bacitracin) and antiviral agents (e.g., acyclovir, docosanol); cytotoxic agents (e.g., DFMO, nitrogen mustard, methotrexate, alkeran, busulfan); counter-irritants/analgesics (e.g., nonsteroidal anti-inflammatory drugs, menthols, capsaicin and derivatives, various alcohols); as well as anti-depressants (e.g., doxepin, trimeprazine, hydroxyzine). Combinations of naloxone or other opiate antagonists with other antipruritic agents including vitamin D, kappa agonists, and irritants such as coal tar derivatives and psoralens are also included in this invention.

The methods and compositions of this invention address the need in the art for an effective anti-pruritic treatment, as set forth above. More specifically, and according to one aspect of this invention, a method of treating pruritus comprises the steps of administering to a patient a composition comprising (i) a therapeutically effective amount of a first compound selected from the group consisting of an opioid receptor antagonist, an opioid receptor mixed agonist/antagonist, and pharmaceutically acceptable salts thereof, and (ii) a second compound useful in the treatment of pruritus, wherein the composition preferably is administered as a topical preparation that includes a multilamellar lipid vesicle delivery system.

According to a further aspect of this invention, a method of treating pruritus comprises the steps of administering to a patient a composition comprising (i) a therapeutically effective amount of a first compound selected from the group consisting of naloxone, naltrexone, nalbuphine, butorphanol, buprenorphine, and pentazocine, and pharmaceutically acceptable salts thereof, and (ii) a second compound useful in the treatment of pruritus.

According to another aspect of this invention, a method of treating pruritus comprises the steps of (i) topically administering to a patient a composition comprising (a) a therapeutically effective amount of a first compound selected from the group consisting of naloxone, naltrexone, and pharmaceutically acceptable salts thereof, (b) a second compound useful in the treatment of pruritus, and (c) optionally a multilamellar lipid vesicle delivery system; and (ii) repeating the step of topically administering the composition until the pruritus is relieved.

According to yet another aspect of this invention, a composition for treating pruritus comprises a first compound selected from the group consisting of naloxone, naltrexone, and pharmaceutically acceptable salts thereof, and a second compound useful in treating pruritus, wherein the composition is in the form of a topical preparation preferably including a multilamellar lipid vesicle delivery system.

According to an additional aspect of this invention, a composition for treating pruritus comprises a first compound selected from the group consisting of opioid receptor antagonists, opioid receptor mixed agonist/antagonists, and pharmaceutically acceptable salts thereof; and a second compound useful in treating pruritus.

According to another aspect of this invention, a method of preparing a composition for use in treating pruritus comprises the steps of combining a first compound selected from the group consisting of naloxone, naltrexone, and pharmaceutically acceptable salts thereof, with a second compound useful in treating pruritus; and mixing said first and second compounds preferably with a multilamellar lipid vesicle delivery system, thereby forming a topical preparation.

It will be apparent to those skilled in the art that only the preferred embodiments have been described by way of exemplification and that there are various modifications which fall within the scope of this invention. These and other aspects of the invention will be discussed in greater detail below.

### DETAILED DESCRIPTION OF THE PREFERRED EMBODIMENTS

While the present invention will be described primarily with respect to a method and composition for treating pruritus, it is to be understood that the features thereof will find applicability to other applications, such as the treatment of other disorders of the skin, scalp, and mucosa. The term “pruritus” as used herein is meant to refer to itching which can range from a mild sensation to an intense sensation of itching pain. The itching may accompany primary skin disease or may be a symptom of systemic disease. The pruritus may be experienced generally over the body, or on specific areas of the skin, scalp, and mucosa.

Briefly, the present invention relates to relieving pruritus using combinations of compounds that impact multiple etiologies, in order to provide more effective and sustained relief. The primary compounds responsible for the antipruritic action of the compositions of this invention are the opioid receptor antagonists, opioid receptor mixed agonist/antagonists, and pharmaceutically acceptable salts thereof. To these compounds, additional compounds are added to impact other pathways related to itching such as local histamine release and inflammation, or the underlying etiology of the clinical condition itself. The compositions and methods according to this invention are suitable for use in relieving pruritus in animals, and are preferably used to treat pruritus in mammals. Particularly preferred compositions and methods are used to treat pruritus or itching in humans.

Non-limiting examples of compounds that are opioid receptor antagonists and opioid receptor mixed agonist/antagonists that may be used according to this invention include, but are not limited to, naloxone, naltrexone, nalbuphine, butorphanol, buprenorphine, and pentazocine, for example. Preferred compounds for use in the compositions and methods of this invention are naloxone and naltrexone, with naloxone being particularly preferred. Preferred pharmaceutically acceptable salts of these compounds include hydrochloride, hydrobromide, hydroiodide, acetate, valerate, oleate, maleate, tartrate, and lactate. Particularly preferred salts include hydrochloride salts.

The additional compounds combined with the opioid receptor antagonists and/or mixed agonist/antagonists are used to treat the underlying cause of the pruritus, and should be selected based on effectiveness in treating the cause of the pruritus in the particular patient. Various compounds that may be useful in treating conditions that cause pruritus include antihistamines, anti-inflammatory corticosteroids, topical antifungals, antibacterials, and antivirals, cytotoxic agents, and counter-irritants/analgesics. Other antipruritic agents within the scope of this invention include anti-depressants, vitamin D, kappa agonists, and irritants such as coal tar derivatives and psoralens. Specific options for the various additional compounds that may be combined with the opioid receptor antagonists and/or mixed agonist/antagonists are set forth below by way of example only.

Antihistamines

These compounds are provided in amounts ranging from about 0.01% to about 5% by weight. Suitable antihistamines for use in compositions according to this invention include, but are not limited to:

cyproheptadine

hydroxyzine

methdilazine

trimeprazine

diphenhydramine

azelastine

brompheniramine

chlorpheniramine

clemastine

promethazine

triprolidine

Anti-Inflammatory Corticosteroids

These compounds are provided in amounts ranging from about 0.01% to about 5% by weight. Suitable anti-inflammatory agents for use in compositions according to this invention include, but are not limited to:

hydrocortisone

prednisone

prednisolone

flurandrenolide

clocortolone

triamcinolone

alclometasone

Antifungals

These compounds are provided in amounts ranging from about 0.01% to about 2% by weight. Suitable antifungals for use in compositions according to this invention include, but are not limited to:

clotrimazole

mycostatin

miconazole

nystatin

oxiconazole

Antibacterials

These compounds are provided in amounts ranging from about 0.01% to about 5% by weight. Suitable antibacterials for use in compositions according to this invention include, but are not limited to:

neomycin

penicillin

ampicillin

bacitracin

Antivirals

These compounds are provided in amounts ranging from about 0.01% to about 5% by weight. Suitable antivirals for use in compositions according to this invention include, but are not limited to:

acyclovir

famciclovir

Antineoplastic/Cytotoxic Agents

These compounds are provided in preferred amounts ranging from about 0.001% to about 3% by weight.

Suitable antineoplastic/cytotoxic agents for use in compositions according to this invention include, but are not limited to:

methotrexate

bleomycin (approximately 1% by weight)

mechlorethamine (nitrogen mustard—about 0.02% by weight)

carmustine (approximately 0.05% by weight)

retinoids (natural and synthetic analogs—about 0.01% to about 1% by weight)

vitamin D (natural and synthetic analogs—about 0.001% to about 0.005% by weight)

DFMO (about 1% to about 5% by weight)

Other antineoplastic/cytotoxic agents useful in the practice of the present invention include but are not limited to:

vincristine

vinblastine

cyclophosphamide

ifosfamide,

hydroxyurea,

busulphan

procarbazine

5-fluorouracil (5-FU).

The appropriate effective dosages for the foregoing antineoplastic/cytotoxic agents are readily determined by one of ordinary skill in the art without undue experimentation.

Counterirritants/Analgesics

These compounds are provided in amounts ranging from about 0.01% to about 10% by weight. Suitable counterirritants/analgesics for use in compositions according to this invention include, but are not limited to:

nonsteroidal anti-inflammatory drugs (including COX-2 inhibitors—about 1% to about 10% by weight)

menthols (about 1% to about 10% by weight)

capsaicin and derivatives (about 0.01% to about 0.1% by weight)

various alcohols

camphor (about 1% to about 15%)

eucalyptus oil (about 0.5% to about 5% by weight)

Antidepressant Agents

These compounds are provided in preferred amounts ranging from about 1% to about 10% by weight. Suitable antidepressant agents for use in compositions according to this invention include, but are not limited to:

doxepin

hydroxyzine

trimeprazine

Antipruritic Agents

These compounds are provided in preferred amounts ranging from about 0.01% to about 5% by weight. Suitable antipruritic agents for use in compositions according to this invention include, but are not limited to:

corticosteroids (see above)

menthols (see above)

vitamin D and analogs (see above)

kappa agonists

emollients (e.g., lanolin)

moisturizers (e.g., urea, aloe, other plant derived products)

The compositions of this invention may also contain opioid receptor antagonists or mixed agonist/antagonists along with compounds including sedatives and tranquilizers, which may be useful in relieving generalized pruritus for which no underlying cause can be determined. The compositions may also contain sequestrants to complex with various salts that may build up as a result of liver or kidney failure that lead to generalized pruritus

In more detail, the compositions of this invention may be formulated as injectables for parenteral administration, as oral and rectal formulations for systemic administration, and as creams, aqueous or non-aqueous suspension, lotions, emulsions, suspensions or emulsions containing micronized particles, gels, foams, aerosols, solids and other suitable vehicles for application to the skin, eyes, lips and mucosa, as suppositories or cream for anal or vaginal administration, and as combinations with bandages, patches, bioadhesives, and dressings for local and topical administration. When the composition is formulated for topical application, a preferred formulation incorporates a multilamellar lipid vesicle delivery system. A particularly preferred multilamellar lipid vesicle delivery system is Crystalip®, which is available from Bioglan Pharma PLC, located in Hertfordshire, UK. This particular formulation is designed for dermal delivery, and enhances the stability of pharmaceutical compounds, such as those used in the composition of this invention, by embedding the compounds in layers of crystalline monoglycerides in an aqueous environment. See U.S. Pat. No. 4,931,284, which is incorporated herein by reference. The compounds are released as the lipid crystals melt at skin temperature after application. This approach increases the bioavailability of the compounds, and also has the benefit of providing an antimicrobial effect. The formulation using Crystalip® can be in the form of a spray, a liquid, or an emulsion, for example.

The methods of this invention provide relief from pruritus, and comprise administering one of the compositions according to this invention to ameliorate or eliminate pruritus, either temporarily or permanently. The compound may be administered in a variety of forms, as described above with respect to the various formulations contemplated for use with the compositions of this invention. The method of administration takes into account the particular formulation being used, i.e., topically applying a cream, lotion, gel, etc. The methods of treating pruritus according to this invention may be used to treat irritation caused by conditions including, for example, inflammation following local infection, blisters, boils, or acute skin injuries such as abrasions, burns, superficial cuts, surgical incisions, toothaches, contusions, inflammatory skin conditions including but not limited to poison ivy, allergic rashes and dermatitis, and any condition that yields a pruritic state or condition.

Formulations and Methods

Effective concentrations of the compositions of this invention, or pharmaceutically acceptable derivatives thereof, are mixed with a suitable pharmaceutical carrier or vehicle for systemic or topical administration. The compositions are provided in an amount effective for reducing the pruritic state or alleviating the condition for which treatment is contemplated. The concentration of the active compounds in the compositions depend on absorption, inactivation, and excretion rates of the active compound, the dosage schedule, and amount administered as well as other factors known to those of skill in the art. For topical and local administration, the dosages are higher, typically by at least about 5 to 10 fold, than the amount delivered when administered systemically or parenterally.

The dosage of the opioid receptor antagonist compound, opioid receptor mixed agonist/antagonist compound, or pharmaceutically acceptable salts thereof, is from about 0.01% to about 5% by weight for antipruritic purposes when administered topically, and is from about 0.1 to about 100 mg/dose for antipruritic purposes when administered parenterally. The dosage of the additional anti-pruritic compound will vary based on the type of compound used, and the appropriate dosage may be readily determined by one skilled in the art.

The compositions may be administered systemically or topically, depending on the formulation used. If the composition is administered topically, a preferred vehicle is a multilamellar lipid vesicle delivery system. However, pharmaceutical carriers or vehicles suitable for administration of the compounds and for use with the methods provided herein include any such carriers known to those skilled in the art to be suitable for the particular mode of administration. In addition, the composition may be formulated with other active ingredients.

1. Systemic Formulations

Typically a therapeutically effective dosage of the composition of this invention is formulated for systemic use to contain from about 0.1 to about 100 mg/dose of an opioid receptor antagonist, mixed agonist/antagonist, or pharmaceutically acceptable salt thereof. The amount of the additional compound used to treat the underlying cause of the pruritus will vary depending on the compound used, and can be readily determined by one skilled in the art. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The formulations of the present invention are provided for administration to humans and animals in unit dosage forms, such as tablets, capsules, pills, powders, granules, sterile parenteral solutions or suspensions, and oral solutions or suspensions, and oil-water emulsions containing suitable quantities of the composition of this invention or pharmacologically acceptable salts thereof.

Oral pharmaceutical dosage forms are either solid or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

Pharmaceutically acceptable carriers utilized in tablets include binders, lubricants, diluents, disintegrating agents, coloring agents, flavoring agents, and wetting agents.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in water or water-in-oil. Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substance used in effervescent granules, to be reconstituted into a liquid oral dosage form, include organic acids and a source of carbon dioxide. Coloring and flavoring agents are used in all of the above dosage forms.

Parenteral administration of the formulations of the present invention includes intravenous, subcutaneous and intramuscular administrations. Preparations for parenteral administration include sterile solutions ready for injection, sterile dry soluble products ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous. Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

The concentration of the pharmaceutically active compound is adjusted so that an injection provides an effective amount to produce the desired pharmacological effect. The exact dose depends on the age, weight and condition of the patient or animal, as is known in the art. The unit-dose parenteral preparations are packaged in an ampoule or a syringe with a needle. All preparations for parenteral administration must be sterile, as is known and practiced in the art. Illustratively, intravenous or intra-arterial infusion of a sterile aqueous solution containing an active compound is an effective mode of administration. Another embodiment is a sterile aqueous or oily solution or suspension containing an active material injected as necessary to produce the desired pharmacological effect.

Pharmaceutical dosage forms for rectal administration are rectal suppositories, capsules and tablets for systemic effect. Rectal suppositories are used herein mean solid bodies for insertion into the rectum which melt or soften at body temperature releasing the pharmacologically and/or therapeutically active ingredients contained in the composition of this invention. Pharmaceutically acceptable substances utilized in rectal suppositories are bases or vehicles and agents to raise the melting point. Examples of bases include cocoa butter (theobroma oil), glycerin-gelatin, carbowax, (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is about 2 to 3 gm. Tablets and capsules for rectal administration are manufactured using the same pharmaceutically acceptable substance and by the same methods as for formulations for oral administration.

2. Topical Formulations

Typically a therapeutically effective dosage of the composition of this invention is formulated to contain a concentration of at least about 0.01% w/w, up to about 5% w/w or more of an opioid receptor antagonist, mixed agonist/antagonist, or pharmaceutically acceptable salt thereof. The amount of the additional compound used to treat the underlying cause of the pruritus will vary depending on the compound used, and can be readily determined by one skilled in the art. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The compositions may be suspended in micronized or other suitable form or may be derivatized to produce a more soluble active product. The form of the resulting composition depends upon a number of factors, including the intended mode of administration and the solubility of the compound in the selected carrier or vehicle. The effective concentration is sufficient for ameliorating the pruritus, and may be empirically determined. The concentration is generally greater than the concentration for systemic administration of the compound.

The resulting mixture may be a solution, suspension, emulsion or the like, and may be formulated as a cream, gel, ointment, emulsion, solution, elixir, lotion, suspension, tincture, paste, foam, aerosol, irrigation, spray, suppository, bandage, or any other formulation suitable for topical or local administration. Preferred modes of administration include topical application to the skin, scalp, eyes, or mucosa. A particularly preferred vehicle according to this invention is a multilamellar lipid vesicle delivery system.

Pharmaceutical and cosmetic carriers or vehicles suitable for administration of the compositions provided herein include any such carriers known to those skilled in the art to be suitable for the particular mode of administration. The active compounds of the compositions are included in the carriers in amounts sufficient to exert a therapeutically useful effect without serious toxic effects on the treated individual. The effective concentration may be determined empirically by testing the compounds using in vitro and in vivo systems, including animal models.

To formulate these compositions, a weight fraction of a composition according to this invention is dissolved, suspended, dispersed or otherwise mixed in a selected vehicle at an effective concentration such that the pruritic condition is relieved or ameliorated. Generally, emollient or lubricating vehicles that help hydrate the skin are more preferred than volatile vehicles, such as ethanol, that dry the skin. Examples of suitable bases or vehicles for preparing compositions for use with human skin are petrolatum, petrolatum plus volatile silicones, lanolin, cold cream (USP), and hydrophilic ointment (USP).

The compositions of this invention relieve pruritus when applied to the skin. The composition may be administered topically to the affected area up to eight times per day, as needed, to provide reduction in and relief from itching. Relief may be temporary or permanent, and may even be evident after a single dose of the composition. When the composition is administered in a form other than a topical preparation, it should be administered in an amount sufficient to provide relief from pruritus that is within safety guidelines established by the FDA. Determining the appropriate amount to administer to a patient is within the skill of the person of ordinary skill in the art.

Solutions of the compositions of this invention intended for topical administration contain an amount of the composition effective to deliver an anti-pruritic amount, typically at a concentration of between about 0.01% w/w to about 5% w/w. The balance of the solution is water, a suitable organic solvent or other suitable solvent or buffer. These compositions that are formulated as solutions or suspensions may be applied to the skin, or may be formulated as an aerosol or foam and applied to the skin as a spray-on. The aerosol compositions typically contain from 25% to 80% w/w, preferably from 30% to 50% w/w, of a suitable propellant. Gel compositions can be formulated by simply admixing a suitable thickening agent to the solution or suspension.

Suitably prepared solutions and suspensions may also be topically applied to the eyes and mucosa. Solutions, particularly those intended for opthalmic use, may be formulated as 0.01%-10% w/w isotonic solutions, pH about 5-7, with appropriate salts, and preferably containing one or more of the compositions herein at a concentration of about 0.1% w/w, up to about 5% w/w or more. Suitable opthalmic solutions are known in the art.

Compositions of solid forms intended for topical application may be formulated as stick-type compositions intended for application to the lips or other parts of the body. Such compositions contain an effective amount of an antipruritic composition according to this invention. The amount of the composition present is typically from about 0.01% w/w to about 5% w/w. The solids also contain from about 40% to 98% w/w, preferably from about 50% to 90% w/w, of emollients. This composition can further contain from 1% to 20% w/w, preferably from 5% to 15% w/w, of a suitable thickening agent, and, if desired or needed, emulsifiers and water or buffers.

In addition, the compositions, and preparations containing the compositions, may also be coated on bandages, mixed with bioadhesives, or included in dressings. Thus, combinations of bandages, bioadhesives, dressings and other such materials and the compositions formulated as described herein are provided. Kits containing these combinations, which may also include compositions containing the above listed agents, are also provided.

3. Methods of Treatment

Compositions for topical use preferably may be applied at least once per day, and if necessary to achieve the desired result, up to eight times per day to the areas of the skin for which treatment is sought. It is understood that the precise treatment regimen depends upon the individual treated and may be ascertained empirically depending upon the formulation, and particularly, the age of the treated individual. Any regimen is acceptable as long as the desired antipruritic effects are achieved without substantial deleterious or sustained undesirable side effects. The compositions may be combined with bandages, bioadhesives and other dressings and applied to the body in combination therewith.

Compositions for systemic use are administered orally or parenterally, depending on the vehicle, in amounts that alleviate the pruritic condition in the animal being treated. The appropriate dosages will depend on various factors, and can be readily determined by a person of skill in the art.

### EXAMPLES

The compositions according to this invention will be described in more detail in the following non-limiting examples.

### Example 1

Topical Formulation of Opioid Receptor Antagonist or Mixed Agonist/Antagonist+Antihistamine Compound

To prepare this composition, combine about 0.25-0.5% by weight of naloxone with about 1-2% by weight of diphenhydramine. These compounds are then mixed, and preferably incorporated into a multilamellar lipid vesicle delivery system to form a topical preparation.

### Example 2

Topical Formulation of Opioid Receptor Antagonist or Mixed Agonist/Antagonist+Anti-Inflammatory Compound

To prepare this composition, combine about 0.5-1% by weight of naltrexone with about 1-2.5% by weight of hydrocortisone. These compounds are then mixed, and preferably incorporated into a multilamellar lipid vesicle delivery system to form a topical preparation.

### Example 3

Topical Formulation of Opioid Receptor Antagonist or Mixed Agonist/Antagonist+Antifungal Compound

To prepare this composition, combine about 0.5-1% by weight of buprenorphine with about 1% by weight of clotrimazole. These compounds are then mixed, and preferably incorporated into a multilamellar lipid vesicle delivery system to form a topical preparation.

### Example 4

Topical Formulation of Opioid Receptor Antagonist or Mixed Agonist/Antagonist+Antibacterial Compound

To prepare this composition, combine about 0.25-0.5% by weight of naloxone with a 3.5 mg/ml neomycin base. Optionally, 10,000 units/gm polymyxin B sulfate and/or 400 units/gm bacitracin may be added to the composition. These compounds are then mixed, and preferably incorporated into a multilamellar lipid vesicle delivery system to form a topical preparation.

### Example 5

Topical Formulation of Opioid Receptor Antagonist or Mixed Agonist/Antagonist+Antiviral Compound

To prepare this composition, combine about 0.5-1% by weight of naltrexone with about 5% by weight of acyclovir. These compounds are then mixed, and preferably incorporated into a multilamellar lipid vesicle delivery system to form a topical preparation.

### Example 6

Topical Formulation of Opioid Receptor Antagonist or Mixed Agonist/Antagonist+Antineoplastic Compound

To prepare this composition, combine about 0.5% by weight of butorphanol with about 0.02% by weight of methotrexate. These compounds are then mixed, and incorporated into a systemic liquid delivery system to form a parenteral preparation.

### Example 7

Topical Formulation of Opioid Receptor Antagonist or Mixed Agonist/Antagonist+Counter-Irritant Compound

To prepare this composition, combine about 0.5-1% by weight of naltrexone with about 0.025-0.1% by weight of capsaicin. These compounds are then mixed, and incorporated into a multilamellar lipid vesicle delivery system to form a topical lotion preparation. The combination of an opioid receptor antagonist or mixed agonist/antagonist with the counter-irritant results in a formulation that produces less than the usual amount of discomfort experienced when applying the counter-irritant alone.

### Example 8

Topical Formulation of Opioid Receptor Antagonist or Mixed Agonist/Antagonist+Antidepressant Compound

To prepare this composition, combine about 0.5-1% by weight of naltrexone with about 5% by weight of doxepin. These compounds are then mixed, and incorporated into a multilamellar lipid vesicle delivery system to form a topical lotion preparation. This formulation antidepressant compound is useful in treating generalized pruritus.

Thus, what has been described is a composition and method for treating pruritus. While the present invention has been described with respect to what are presently considered to be the preferred embodiments, it is to be understood that the invention is not limited to the disclosed embodiments. To the contrary, the invention is intended to cover various modifications and equivalent arrangements included within the spirit and scope of the appended claims. Therefore, the scope of the following claims is to be accorded the broadest interpretation so as to encompass all such modifications and equivalents.

We claim:

1. A composition for use in treating pruritus, comprising:
a first compound selected from the group consisting of naloxone, naltrexone, and pharmaceutically acceptable salts thereof; and
a second compound useful in treating an underlying cause of the pruritus, wherein the composition is in the form of a topical preparation and optionally including a multilamellar lipid vesicle delivery system.

2. The composition of claim 1, wherein the pharmaceutically acceptable salts are selected from the group consisting of hydrochloride, hydrobromide, hydroiodide, acetate, valerate, oleate, maleate, tartrate, and lactate.

3. The composition of claim 2, wherein the pharmaceutically acceptable salt is hydrochloride.

4. The composition of claim 1, wherein the second compound useful in treating the underlying cause of the pruritus is selected from the group consisting of antihistamines, anti-inflammatory agents, antifungal agents, antibacterial agents, antiviral agents, antineoplastic agents, counter-irritants, antidepressants, and antipruritic agents.

5. The composition of claim 1, wherein the first compound is present in an amount of from about 0.01% to about 5% by weight.

6. A composition for use in treating pruritus, comprising:
a first compound selected from the group consisting of opioid receptor antagonists, opioid receptor mixed agonist/antagonists, and pharmaceutically acceptable salts thereof, and
a second compound useful in treating an underlying cause of the pruritus.

7. The composition of claim 6, wherein the first compound is selected from the group consisting of naloxone, naltrexone, nalbuphine, butorphanol, buprenorphine, and pentazocine.

8. The composition of claim 6, wherein the pharmaceutically acceptable salts are selected from the group consisting of hydrochloride, hydrobromide, hydroiodide, acetate, valerate, oleate, maleate, tartrate, and lactate.

9. The composition of claim 6, wherein the second compound useful in treating the underlying cause of the pruritus is selected from the group consisting of antihistamines, anti-inflammatory agents, antifungal agents, antibacterial agents, antiviral agents, antineoplastic agents, counter-irritants, antidepressants, and antipruritic agents.

10. The composition of claim 6, wherein the first compound is present in an amount of from about 0.01% to about 5% by weight.

11. The composition of claim 6, wherein the first compound is present in an amount of from about 0.1 to about 100 mg/dose.

12. The composition of claim 6, wherein the dose is in the form of a capsule, tablet, or liquid.

13. The composition of claim 12, wherein the liquid dose is in the form of an emulsion, solution, or suspension, and may further contain a volatile diluent selected from the group consisting of alcohols and glycols.

14. A composition for use in treating pruritus, comprising:
a therapeutically effective amount of a first compound selected from the group consisting of naloxone, naltrexone, nalbuphine, butorphanol, buprenorphine, and pentazocine, and pharmaceutically acceptable salts thereof, and
a second compound useful in the treatment of an underlying cause of the pruritus.

15. The composition of claim 14, wherein the pharmaceutically acceptable salts are selected from the group consisting of hydrochloride, hydrobromide, hydroiodide, acetate, valerate, oleate, maleate, tartrate, and lactate.

16. The composition of claim 14, wherein the second compound useful in treating the underlying cause of the pruritus is selected from the group consisting of antihistamines, anti-inflammatory agents, antifungal agents, antibacterial agents, antiviral agents, antineoplastic agents, counter-irritants, antidepressants, and antipruritic agents.

17. The composition of claim 14, wherein the first compound is present in an amount of from about 0.01% to about 5% by weight.

18. The composition of claim 14, wherein the first compound is present in an amount of from about 0.1 to about 100 mg/dose.

19. The composition of claim 14, wherein the dose is in the form of a capsule, tablet, or liquid.

20. The composition of claim 19, wherein the liquid dose is in the form of an emulsion, solution, or suspension, and may further contain a volatile diluent selected from the group consisting of alcohols and glycols.

21. A method for treating pruritus, comprising the steps of:
administering to a patient a composition comprising
a therapeutically effective amount of a first compound selected from the group consisting of an opioid receptor antagonist, an opioid receptor mixed agonist/antagonist, and pharmaceutically acceptable salts thereof, and
a second compound useful in the treatment of an underlying cause of the
pruritus,
wherein the composition is administered as a topical preparation that includes a multilamellar lipid vesicle delivery system.

22. The method of claim 21, wherein the first compound is selected from the group consisting of naloxone, naltrexone, nalbuphine, butorphanol, buprenorphine, and pentazocine.

23. The method of claim 21, wherein the pharmaceutically acceptable salts are selected from the group consisting of hydrochloride, hydrobromide, hydroiodide, acetate, valerate, oleate, maleate, tartrate, and lactate.

24. The method of claim 21, wherein the second compound useful in treating the underlying cause of the pruritus is selected from the group consisting of antihistamines, anti-inflammatory agents, antifungal agents, antibacterial agents, antiviral agents, antineoplastic agents, counter-irritants, antidepressants, and antipruritic agents.

25. The method of claim 21, wherein the composition is suitable for application to skin, scalp and hair.

26. The method of claim 21, wherein the composition in provided in a vehicle selected from the group consisting of creams, ointments, gels, pastes, shampoos, solutions, topical patches, adhesive dressings, and lotions.

27. The method of claim 21, wherein the composition is applied from one to about eight times per day.

28. The method of claim 21, wherein the composition further comprises one or more of wetting agents, emulsifying agents, and suspending agents.

29. The method of claim 21, wherein the composition is suitable for application to mucous membranes.

30. The method of claim 21, wherein the pruritus is caused by an allergic reaction.

31. The method of claim 21, wherein the pruritus is caused by a topical infection.

32. A method for treating pruritus, comprising the steps of:
administering to a patient a composition comprising
a therapeutically effective amount of a first compound selected from the group consisting of naloxone, naltrexone, nalbuphine, butorphanol, buprenorphine, and pentazocine, and pharmaceutically acceptable salts thereof, and
a second compound useful in the treatment of an underlying cause of the pruritus.

33. The method of claim 32, wherein the pharmaceutically acceptable salts are selected from the group consisting of hydrochloride, hydrobromide, hydroiodide, acetate, valerate, oleate, maleate, tartrate, and lactate.

34. The method of claim 32, wherein the second compound useful in treating the underlying cause of the pruritus is selected from the group consisting of antihistamines, anti-inflammatory agents, antifungal agents, antibacterial agents, antiviral agents, antineoplastic agents, counter-irritants, and antipruritic agents.

35. The method of claim 32, wherein the composition is suitable for topical application.

36. The method of claim 32, wherein the composition is suitable for application to skin, scalp and hair.

37. The method of claim 32, wherein the composition in provided in a vehicle selected from the group consisting of creams, ointments, gels, pastes, suppositories, enemas, shampoos, solutions, topical patches, adhesive dressings, lotions, tablets, and capsules.

38. The method of claim 32, wherein the composition is applied topically from one to about eight times per day.

39. The method of claim 32, wherein when the composition is applied topically, it further comprises one or more of wetting agents, emulsifying agents, and suspending agents.

40. The method of claim 32, wherein the composition is suitable for application to mucous membranes.

41. The method of claim 32, wherein the composition is suitable for oral administration.

42. The method of claim 32, wherein the composition is suitable for parenteral administration.

43. The method of claim 32, wherein the pruritus is caused by an allergic reaction.

44. The method of claim 32, wherein the pruritus is caused by a topical infection.

45. A method for treating pruritus, comprising the steps of:
topically administering to a patient a composition comprising
a therapeutically effective amount of a first compound selected from the group consisting of naloxone, naltrexone, and pharmaceutically acceptable salts thereof,
a second compound useful in the treatment of an underlying cause of the pruritus, and
a multilamellar lipid vesicle delivery system; and
repeating the step of topically administering the composition until the pruritus is relieved.

46. The method of claim 45, wherein the pharmaceutically acceptable salts are selected from the group consisting of hydrochloride, hydrobromide, hydroiodide, acetate, valerate, oleate, maleate, tartrate, and lactate.

47. The method of claim 45, wherein the second compound useful in treating the underlying cause of the pruritus is selected from the group consisting of antihistamines, anti-inflammatory agents, antifungal agents, antibacterial agents, antiviral agents, antineoplastic agents, counter-irritants, antidepressants, and antipruritic agents.

48. The method of claim 45, wherein the composition is suitable for application to skin, scalp and hair.

49. The method of claim 45, wherein the composition in provided in a vehicle selected from the group consisting of creams, ointments, gels, pastes, suppositories, enemas, shampoos, solutions, topical patches, adhesive dressings, and lotions.

50. The method of claim 45, wherein the composition is applied topically from one to about eight times per day.

51. The method of claim 45, wherein the composition further comprises one or more of wetting agents, emulsifying agents, and suspending agents.

52. The method of claim 45, wherein the composition is suitable for application to mucous membranes.

53. The method of claim 45, wherein the pruritus is caused by an allergic reaction.

54. The method of claim 45, wherein the pruritus is caused by a topical infection.

55. A method for preparing a composition for use in treating pruritus, comprising the steps of:
combining a first compound selected from the group consisting of naloxone, naltrexone, and pharmaceutically acceptable salts thereof, with a second compound useful in treating an underlying cause of the pruritus; and optionally
mixing said first and second compounds with a multilamellar lipid vesicle delivery system, thereby forming a topical preparation.

56. A composition for use in relieving pruritus including an opioid receptor antagonist, mixed agonist/antagonist, or pharmaceutically acceptable salt thereof, the improvement comprising:
a second compound useful in treating an underlying cause of the pruritus; and
a formulation comprising a topical preparation including a multilamellar lipid vesicle delivery system.
